(19) United States
(12) Patent Application Publication
(10) Pub. No.: US 2004/0023845 A1
(43) Pub. Date: Feb. 5, 2004

(54) Method for identifying CCR5 receptor antagonists by measuring residency time

(76) Inventors: Patrick Kari Dorr, (US); .Manoussos Perros, (US); Graham Anthony Rickett, (US)

Correspondence Address: AGOURON PHARMACEUTICALS, INC., 10350 NORTH TORREY PINES ROAD, LA JOLLA, CA, 92037, US

(21) Appl. No.: 10/442,358

(22) Filed: May 20, 2003

Related U.S. Application Data
(60) Provisional application No. 60/386,996, filed on Jun. 7, 2002.

(30) Foreign Application Priority Data
May 23, 2002 (GB) 0211923.8

(51) Int. Cl. A61K 31/00; C12Q 1/70; G01N 33/53; G01N 33/567

(52) U.S. Cl. 514/1; 435/5; 435/7.2

(57) ABSTRACT

The present invention relates to the use of an assay that measures receptor residence time of a ligand on its receptor in vitro for the identification of a ligand for that receptor predicted to be efficacious in vivo in the treatment of a disease that responds to modulation of that receptor's natural function.

This application claims priority from United Kingdom application number 0211923.8, filed May 23, 2002, and U.S. Provisional application No. 60/386,996, filed Jun. 7, 2002, and incorporates each application by reference in its entirety.

### BACKGROUND OF THE INVENTION

The present invention relates to the use of an assay to determine receptor occupancy as a means to identify ligands predicted to be clinically efficacious. In particular, the present invention relates to the identification of ligands of the CCR5 receptor using a long receptor residency time as an indicator of anti-viral, specifically anti-HIV, activity.

Producing a blockbuster medicine is becoming increasingly difficult, with more and more money being spent on research and development while the number of new drugs reaching the patient rarely matches the investment. Attrition of potential new drugs typically results from safety issues, and compound survival rates from discovery to the clinic has become one of the most pressing issues facing the pharmaceutical industry.

Costs up to clinical phase can be around $20 million. For every ten to fifteen compounds, only one survives the clinical phase. Once a compound has shown sufficient in vitro activity and has already been tested on animals, the costs rise sharply.

Phase I tests on healthy volunteers cost an average of $20 million per studied compound and around one in two compounds fail. Phase II tests in humans to study correct dosage cost, on average, $40 million per compound and around one in two fail. Phase III double-blind trials around the world in large patient populations to determine efficiency against placebo or other frequently used product costs an average of $560 million. Phase III trials at $560 million is 70% of a product's total cost and there is, on average, only a 58% chance of survival.

The final stage for a new drug is filing for approval or registration. Every patient of the thousands studied has an average file of fifty pages and costs are in the region of $160 million. Increasingly, strict and severe guidelines mean only approximately 74% of new drugs are approved.

Therefore, attriting those compounds least likely to survive, before they are progressed to the clinical stage, is critical. Pharmacokinetic and drug metabolism studies, as well as drug safety evaluation, clinical precedence and knowledge about the safety of a molecule or mechanism, all provide valuable and essential data when evaluating a compound for its potential in the clinic. However, methods to predict the potential clinical efficacy of a compound early in development are clearly desired.

Although HIV-induced AIDS (Acquired Immunodeficiency Syndrome) was first described almost two decades a go, it continues to be a disease of enormous proportions. It is estimated that there were more than 40 million HIV-infected people worldwide by the end of 2002 (UNAIDS/WHO. AIDS epidemic update: Dec 2002 http://www.unaids.org/worldaidsday/2002/press/epupdate.html).

Human Immunodeficiency Virus Type 1 (HIV-1) attacks cells in the immune system, causing the body to lose the ability to fight off infections and diseases. During the course of infection, CD4 T-cells (white blood cells that fight infection) are disabled and killed as their numbers decline. Infection with HIV-1 leads, in the vast majority of cases, to progressive disease and ultimately, AIDS and death.

Unfortunately, even with the most potent drug treatments now available, to suppressing HIV-1 remains an incredibly frustrating challenge, in part due to the fact that HIV-1 is one of the most rapidly mutating viruses ever encountered. Recent developments have resulted in the use of a potent three-drug cocktail, known as highly active antiretroviral therapy (HAART), to reduce HIV-1 replication. HAART currently targets two different stages in the virus life cycle and consists of two or three inhibitors of the virus's reverse transcriptase combined with at least one inhibitor of the viral protease. This combination antiretroviral therapy results in a dramatic reduction in viral load, decreasing the rate of CD4 cell decline and the progression to AIDS in many patients. However, the side effect profiles for several of these drugs make long-term adherence difficult and emergence of drug resistance a serious problem. Furthermore, sensitive testing shows that even the best treatments cannot completely suppress viral replication. Because HIV-1 mutates at an incredibly fast rate, any viral replication allows drug-resistant mutants a chance to appear. Once HIV-1 mutates, few viable treatment options remain for an infected individual.

Currently most patients will eventually fail combination therapies of drugs of the existing three classes, either due to intolerance or resistance (or a combination of both) and therefore there remains a high medical need for better tolerated and conveniently administered agents to treat HIV-1/AIDS. One new approach is via host cell entry inhibition, an example of which is through blockade of the CCR5 co-receptor.

To invade the human cell, HIV-1 must bind to the cluster of differentiation (CD) CD4 receptor and a chemokine co-receptor (either CCR5 or CXCR4) located on the CD4 T cell surface. Binding to the CD4 receptor alone is not sufficient to render cells susceptible to infection by HIV-1. Macrophage tropic (M-tropic) HIV isolates, irrespective of subtype, predominantly use the β-chemokine receptor CCR5, and are referred to as R5 viruses. R5 viruses are preferentially transmitted and predominate in early asymptomatic disease. A switch in chemokine receptor utilization from CCR5 to CXCR4 may indicate the transition from asymptomatic infection to AIDS. It is not known whether the co-receptor switch contributes to or results from disease progression.

The inhibition via CCR5 is a particularly attractive drug target for two reasons. Firstly, as mentioned above, the viruses that establish a new infection are generally CCR5-tropic. Thus, successful inhibition of infection via CCR5 might significantly decrease the probability of transmission. Secondly, individuals homozygous for a naturally occurring 32 base-pair deletion in the CCR5 gene (CCR5Δ32) and thus lacking this receptor are apparently immunologically normal. These individuals are also resistant to infection by R5 strains of HIV and people heterozygous for this CCR5 defect exhibit a slower progression to AIDS and death. The frequency of the different genetic polymorphisms varies between populations. CCR5Δ32 is most prevalent in northern Europe (allelic frequency of 16%), less common in individuals from southern Europe (4%) and is extremely rare in African populations. Approximately 1% of Caucasians are homozygous for CCR5Δ32 and effectively represent human equivalents to experimental “knock-out” animals.

Chemokine receptors are seven membrane-spanning molecules found on the surface primarily of cells in the immune system. When these receptor molecules bind their ligands, i.e., chemokines, the end result is to recruit cells of the immune system to the site of tissue damage or disease.

CCR5 (CC chemokine receptor 5) is the major co-receptor for macrophage-tropic (M-tropic) HIV-1 strains and also appears to be the major co-receptor for microglial tropic HIV-1 primary isolates. Putative physiological ligands of the CCR5 receptor are the chemokines Macrophage Inflammatory Protein-1 (MIP-1α), MIP-1β, and Regulated upon Activation, Normal T Expressed, and Secreted (RANTES). Although a single domain, consisting of the second extracellular loop, appears to be predominantly responsible for chemokine binding, multiple extracellular domains of CCR5 are involved in HIV-1 co-receptor activity.

During the M-tropic phase of HIV infection, the virus favors macrophages, which it invades by binding (through its gp120 protein) to the molecules CD4 and CCR5 on the macrophage surface. Eventually, however, HIV-1 can become dual-tropic. Such strains produce gp120 molecules capable of recognizing the CXCR4 protein on CD4-bearing T-cells. During this phase HIV-1 may infect both macrophages and T-cells. Still later, the bulk of the viral population may switch its preference to the CXCR4 receptor and become T-tropic. T-tropic viruses readily destroy infected T-cells, contributing to the collapse of the immune system and the onset of AIDS. Alternatively, some viruses, such as certain strains of HIV-2, could attach to CXCR4, leading quickly to AIDS.

As the absence of a biologically functional CCR5 receptor does not appear detrimental to the host, there are hopes that agents can be developed to mimic the protective effect of the CCR5 deletion. Possibilities include blocking the wild-type expressed receptors with suitably constructed agonists or antagonists to these molecules. The challenge remains to ensure that analogues are potent, non-toxic, exhibit good pharmacologic profiles and can be administered orally.

Compounds that are potent antagonists of CCR5 have been identified, for example in patent applications WO 00/38680, WO 00/39125, and EP 1013276, the disclosures of which are incorporated herein by reference. However, it has become apparent in our work that high binding affinity of CCR5 antagonists shown in pre-clinical studies does not always translate into equally high antiviral activity, especially into good clinical efficacy, of the compounds. Indeed, the prediction of clinical efficacy using known pre-clinical methods remains a problem, resulting in the unnecessary and costly use of potentially ineffective compounds in clinical trials.

### SUMMARY OF THE INVENTION

The present invention is based on the observation that the offset time, or functional occupancy, of a receptor by a ligand or modulator provides a guide to the in vivo efficacy and pharmacodynamics of the ligand or modulator. In particular, the Applicant has found that compounds with very similar binding affinity to a receptor show marked differences in their residency time on the receptor, which may translate to improved antiviral potency. Thus, the aim of the present invention is to provide a method whereby the potential for favourable pharmacodynamics and improved potency, and therefore clinical efficacy of a compound, may be predicted more accurately from pre-clinical data.

In one aspect, the present invention resides in the use of an assay that measures receptor residence time of a ligand on its receptor in vitro for the identification of a ligand for that receptor predicted to be efficacious in vivo in the treatment of a disease that responds to modulation of that receptor's natural function.

Expressed in another way, the present invention relates to a method for identifying ligands with high potency and/or clinical efficacy for a disease that responds to modulation of a receptor's natural function which comprises measuring their residence time on the receptor and selecting ligands on the basis of the desired residence time.

Advantageously, the receptor residence time is at least 1 hour, at least 3 hours, at least 6 hours or, more advantageously, at least 9 hours. In particular, the longer the functional occupancy of a ligand on a receptor, the longer the clinical effect of that ligand, thereby reducing the number of doses that would need to be administered to a patient.

The ligands identified by the present invention may be agonists for the receptor but, preferably, are antagonists. Preferably the receptor is a G-protein coupled receptor, most preferably the chemokine receptor CCR5.

The receptor residence time (or clinical efficacy) of a ligand may be a predictor of anti-viral activity, preferably anti-HIV activity.

In particular, the current invention provides a method to select CCR5 antagonists, which will show potent antiviral activity and clinical efficacy, by measuring the residence time of the antagonists on CCR5 and selecting ligands that have a long residence time.

In another aspect, the present invention relates to a research method comprising measuring the receptor residence time of each of a plurality of ligands for that receptor, and selecting for further research at least one ligand whose residence time is longer than that of at least one other ligand.

In a further aspect, the present invention resides in a research method comprising contacting a plurality of ligands for a given receptor with that receptor, measuring the receptor binding affinity and receptor residence time of each ligand, assigning to each ligand a rank value which is the product of its measure binding affinity and its receptor residence time, and selecting for further investigation one or more ligands having a rank value greater than a chosen cut-off rank value.

Potency and/or clinical efficacy relates to the functional effect a ligand exhibits while resident on its target receptor. This may be any effect an agonist or an antagonist to a receptor may have, for example the inhibition of viral entry into T-cells. In a preferred embodiment, the effect is an antiviral effect, preferably an anti-HIV effect.

The desired residence time may be short for some conditions, or long for others. In a preferred embodiment of the invention, the desired residence time is at least 1 hour, preferably at least 3 hours, even more preferably at least 6 hours, most preferably at least 9 hours.

In yet a further aspect, the invention relates to a ligand selected by the method of the invention.

### Definitions

As used herein, a “ligand” for a receptor means any compound that binds to the receptor and thereby modulates the natural function of the receptor. The term includes, but is not limited to, peptide, modified peptide, polypeptide, protein and small molecule ligands, such as synthetic chemical compounds, naturally occurring compounds or small organic molecules. Alternatively, the ligand may be an antibody or antibody fragment, or a nucleic acid or nucleic acid derived material.

As used herein, “residence time” means the average time a ligand spends bound to its receptor, i.e., the average time between the “on” and “off” states of the ligand with respect to its receptor. Expressed in another way, the term refers to the time during which the receptor is in a state that does not allow a natural ligand, such as a chemokine, or invading molecule, such as HIV glycoprotein, to bind as a result of ligand binding.

Residence time is also referred to as “receptor occupancy” and may be determined by any method that measures the off-rate, dissociation rate, or off-set rate of a ligand. A typical method comprises the steps of incubating a labelled ligand with an appropriate receptor, for example cells expressing the receptor or membrane preparations derived from such cells, until equilibrium is reached. Excess unlabelled ligand is then added and the amount of labelled ligand bound to the receptor is measured at desired time intervals. The label may be any detectable tag that does not influence the binding of the ligand to its receptor. Preferably, a radiolabel such as ³H, ¹⁴C, ³²P or ¹²⁵I is incorporated into the ligand, most preferably ³H.

The term “receptor” is to be understood broadly and may relate to any molecule comprising a binding site for a ligand as defined above, including, but not limited to, receptors, enzymes, ion channels, adhesion molecules, antibodies. In a preferred embodiment, the receptor is a receptor found in or on a cell, preferably a mammalian cell, even more preferably a human cell. In another preferred embodiment, the receptor is a cell surface receptor, more preferably a G-protein coupled receptor, even more preferably a chemokine receptor. In another preferred embodiment, the receptor for this invention is the CCR5 chemokine receptor.

The term “binding affinity” relates to the binding of a ligand that may be determined by traditional binding assays well known in the art, such as a competition assay. Typically, a membrane preparation from cells expressing the receptor of interest will be incubated with a known, labelled ligand for this receptor, using a concentration of the labelled ligand that gives about 50% of the total possible binding. In parallel assay tubes, an unlabelled, competing test ligand is included in varying amounts, in order to measure the ability of the test compound to compete for the binding with the labelled ligand. Competition curves are then generated, plotting the concentration of the test ligand used along the x-axis, and the amount of label bound along the y-axis. From such experiments, the K_{D}, the IC₅₀ (i.e., the concentration of competing ligand which displaces 50% of the labelled ligand), or the IC₉₀ (i.e., the concentration of competing ligand which displaces 90% of the labelled ligand) value may be calculated. There are many ways of expressing the binding affinity of ligands with which the skilled person will be well versed to calculate and which may be found in any basic pharmacology text book.

“Chemokine receptor” refers to receptors for a large family of proteins which are chemotactic cytokines, i.e., have the ability to attract leukocytes as leukocyte chemotactic factors. Chemokines share certain important structural features, and bind to families of receptors most of which belong to the G-protein coupled receptor superfamily. Chemokines and their receptors are central to the pathophysiology of inflammatory and infectious diseases and agents that are active in modulating, preferably antagonising, the activity of chemokines and their receptors, are useful in the treatment of such inflammatory and infectious diseases.

“CCR5” refers to the chemokine receptor which is the cellular receptor for the β-chemokines RANTES, MIP-1α and MIP-1β. CCR5 has also been identified as being an important receptor in HIV infection, binding the HIV envelope glycoprotein gp120. Background teachings on CCR5 may be found in WO 97/32019, the disclosure of which is incorporated herein by reference.

“HIV” refers to human immunodeficiency virus, presumed to be the agent causing AIDS. There is a wealth of literature on this subject; one of the earliest references to HIV, originally named HTLV-III, is made in Ratner et al, Nature 313, 277-284, 1985.

### DETAILED DESCRIPTION OF THE INVENTION

The present invention will be further described, by way of example, with reference to the following figures:

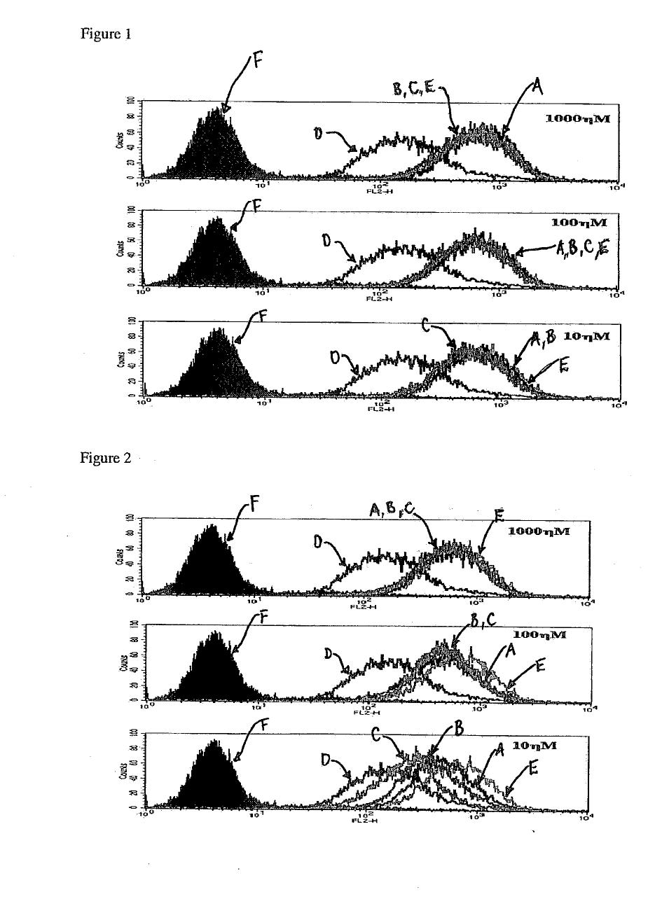

FIG. 1 shows the FACS fluorescent overlays for RANTES-mediated CCR5 internalisation in 300.19 cells and the inhibition of internalisation by Compound B at various concentrations: without compound removal (A), following compound removal by washing (B) and following removal and 1.5 hour rolling incubation (C). The fluorescence shift due to RANTES-mediated CCR5 internalisation is depicted by trace D, and the vehicle control (no compound or RANTES, i.e., total CCR5) is depicted by trace E. Isotype control is depicted by trace F.

FIG. 2 shows the FACS fluorescent overlays for RANTES-mediated CCR5 internalisation in 300.19 cells and the inhibition of internalisation by Compound C at various concentrations: without compound removal (A), following compound removal by washing (B) and following removal and 1.5 hour rolling incubation (C). The fluorescence shift due to RANTES-mediated CCR5 internalisation is depicted by trace D, and the vehicle control (no compound or RANTES, i.e., total CCR5) is depicted by trace E. Isotype control is depicted by trace F.

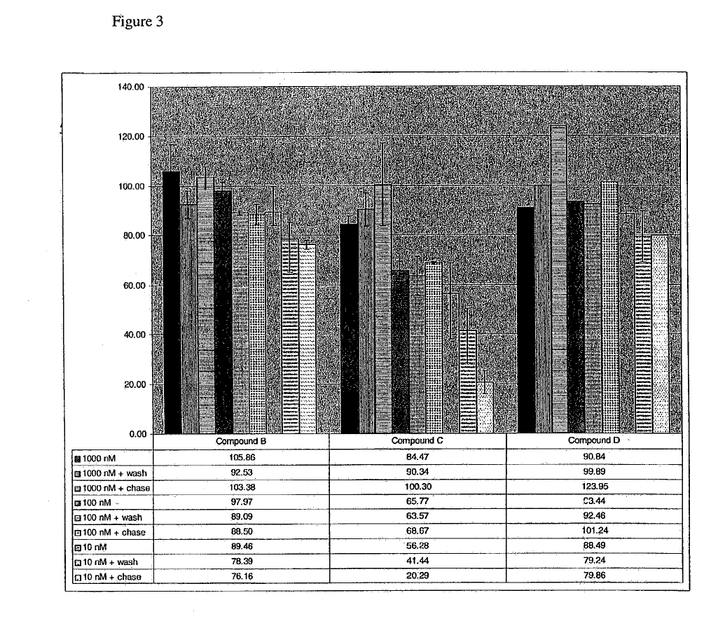

FIG. 3 is a graph showing the relative inhibition (% relative to vehicle control) of RANTES-induced internalisation of CCR5 by Compounds B, C and D, when incubation is followed by a wash step (+wash) or 1.5 hours incubation in the absence of compound (+chase).

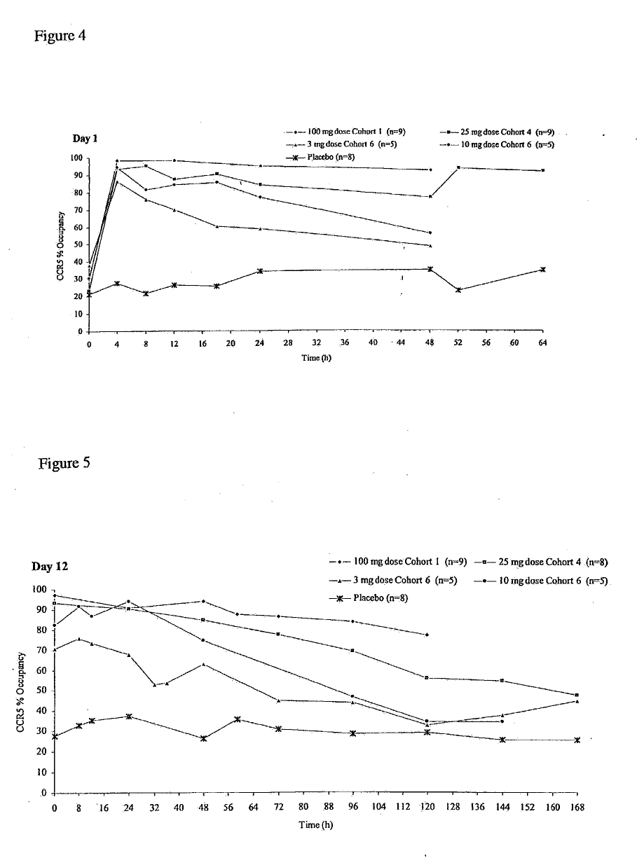

FIG. 4 is a graph showing the mean Day 1% CCR5 occupancy on CD4 T cells for Cohorts 1, 4 and 6 subjects receiving placebo or a single 100 mg, 25 mg and 3 or 10 mg oral dose of Compound A, respectively, administered as a solution.

FIG. 5 is a graph showing the mean day 12%CCR5 occupancy on CD4 T cells for cohorts 1, 4 and 6 subjects receiving placebo or 100 mg, 25 mg and 3 or 10 mg b.i.d. oral doses of Compound A, respectively, on Days 3 to 12, administered as a solution.

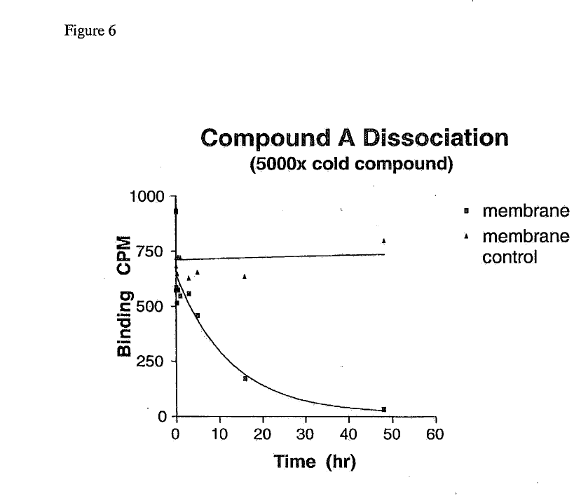

FIG. 6 is a dissociation curve for Compound A.

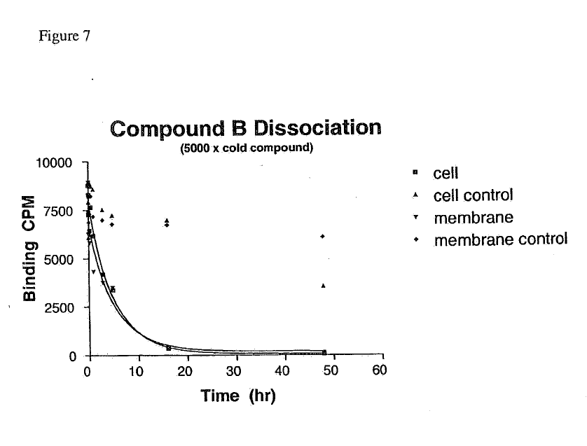

FIG. 7 is a dissociation curve for Compound B.

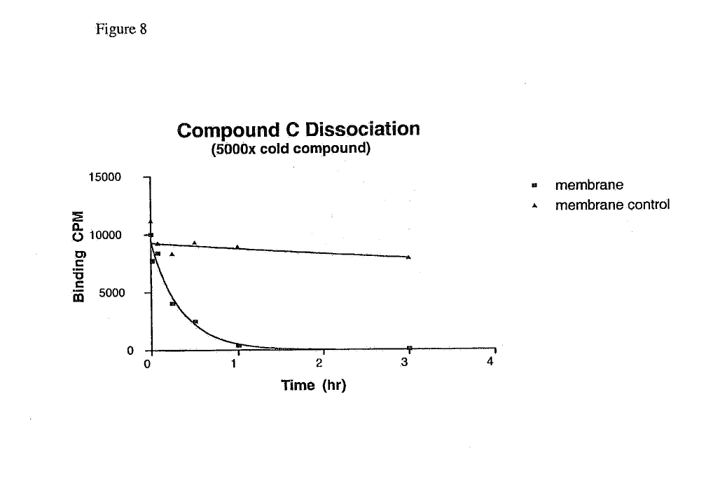

FIG. 8 is a dissociation curve for Compound C.

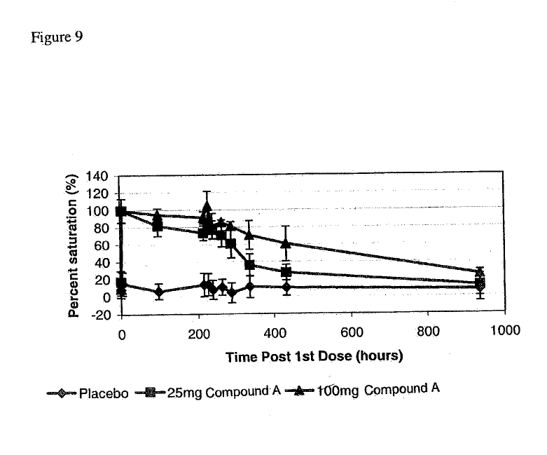

FIG. 9 is a graph showing CCR5 receptor saturation over time in asymptomatic HIV seropositive male subjects administered with Compound A 25 mg, 100 mg or placebo.

The invention is exemplified with the use of four compounds, where:

Compound A is Example 4 in WO 01/90106;

Compound B is Example 34 in WO 01/38680;

Compound C is not disclosed; and

Compound D is Example 33 in PCT/IB/03/01220.

### EXAMPLE 1

### Binding of Compounds to CCR5

Compounds were tested in an assay for CCR5 affinity following the procedures disclosed in Combadiere et al, J Leukoc. Biol. 60, 147-152 (1996). Compounds A to D all had an IC₅₀ for CCR5 binding of less than 10 nM (data not shown).

### EXAMPLE 2

### Antagonist-Dependent Inhibition of Chemokine-Mediated CCR5 Internalisation

1. Functional Occupancy of CCR5 on 300.19 Cells as Measured by Dynamic Inhibition of RANTES-Mediated CCR5 Internalisation by FACS Analysis

Physical occupancy of the CCR5 receptor by antagonists can be measured using radioligand dissociation studies. The functional occupancy of antagonists on the CCR5 receptor on whole cells can be assessed by measuring antagonist-dependent inhibition of cognate chemokine-mediated receptor internalisation. These measurements can be made using fluorescence activated cell sorting (FACS) technology. The prolongation of either the inhibition of chemokine-mediated CCR5 internalisation, following the removal of antagonist by cell washing, is a measurement of the functional occupancy of CCR5.

Methods

All assays/reagents were performed/used at room temperature unless otherwise stated.

Cell Culture: 300.19 cells (mouse pre-B cell line, recombinantly expressing human CCR5) were cultured in 75 cm² cell culture flasks in growth medium at 37° C. for 2-3 days in the humidified 5% (v/v) CO₂ incubator. For cell passage, routine splits from 1×10⁵ to 2×10⁵ of the suspension cells were performed.

Cell Preparation: Cells from each 75 cm² flask used were centrifuged at 1500 rpm on a bench top centrifuge for 5 minutes. Pelleted cells were resuspended in a minimal volume of RPMI (10% FBS) culture medium (see Materials), counted on a Cedex cell counter (see Materials), and adjusted to a cell density of 5×10⁶/ml by dilution in the same medium.

Antagonist, Antibody, and Chemokine Preparation: CCR5 antagonists were dissolved in 100% dimethyl sulphoxide (DMSO) to a concentration of 1 mM and diluted in RPMI (10% FBS) cell culture medium to enable testing in the FACS assays at concentrations from 10 nM to 1000 nM in situ.

Anti-CCR5 antibody (2D7—see Materials) was diluted 1:10 in 0.5% BSA/PBS. Antibody IgG2a (isotype control for the assay—see Materials) was used diluted 1:10 in 0.5% BSA/PBS. The anti-2D7 phyco-erythrin (PE)-labeled goat anti-mouse secondary antibody (see Materials) was used at a 1:20 dilution in 0.5% BSA/PBS.

The cognate agonist chemokine ligand for CCR5, Regulated on Activation, Normal T-Expressed and Secreted (RANTES), was solubilised in PBS. Lyophilised material (10 μg—see Materials) was resuspended in PBS to a concentration of 100 μM. Dilutions were then made in RPMI (10% FBS) to give a final concentration of 1000 nM (100 nM final concentration in assay).

300.19 cells (100 μl) at 5×10⁶ cells/ml were added to each assay tube. CCR5 antagonists (10 μl) or vehicle (RPMI (10% FBS)) were added to appropriate assay tubes to enable profiling over concentrations varying from 10-1000 nM in situ. The tubes were incubated at 4° C. for 45 minutes to allow antagonist association to the cells. To investigate prolongation of CCR5 occupancy by antagonists, samples were either left with antagonist present or centrifuged (1500 rpm in a bench top centrifuge) and washed twice in 1000 μl RPMI (10% FBS). Half of the washed samples were resuspended in 1000 μl of the same media and placed on a rolling platform for one and a half hours before being processed through the FACS assay. The other half of the washed samples, together with the unwashed samples (i.e., antagonist present), were processed immediately.

RANTES (10 μl) was added to the samples followed by incubation at 37° C. for 45 minutes to provoke CCR5 internalisation. The samples were centrifuged (1500 rpm in a bench top centrifuge) and washed twice in 0.5%BSA/PBS. Washed samples were resuspended in 40 μl of the same buffer. Then, 2D7 antibody or isotype control (10 μl) was added to the samples followed by incubation for 45 minutes at 4° C. to enable antibody binding to CCR5.

The samples were then washed once in 0.5%BSA/PBS followed by the addition of 75 μl phyco-erythrin (PE)-goat anti-mouse secondary antibody (1:20 dilution in 0.5% BSA/PBS) with incubation at 4° C. for 45 minutes in the dark to enable binding. The samples were subsequently centrifuged (1500 rpm on a bench top centrifuge for 5 minutes), washed twice in 100 μl 0.5% BSA/PBS, and resuspended in 1000 μl of 1% formaldehyde/PBS for fixing. The fixed cells were processed in a Becton Dickinson FACScalibur, to measure the fluorescence levels on the cells in each assay to assess antagonist-dependent inhibition of RANTES-mediated CCR5 internalisation and consequently functional CCR5 occupancy. The FACScalibur assays were run using Cell Quest software according to the supplier's instruction manual, using excitation/emission wavelengths of 488 nm/530 nm respectively.

Materials

Culture medium: 1×500 ml RPMI-1640 medium with NaHCO₃ without L-glutamine—Gibco BRL (cat no: 31870-025); 5 ml 200 mM L-Glutamine—Gibco BRL (cat no: 25030-024); 5 ml Penicillin/Streptomycin (10,000U/ml Pen, 10,000 μg/ml Strep)—Gibco BRL (cat no: 15140-122); 50 ml foetal bovine serum (FBS)—Sigma Aldrich (cat no: F7524); 5 ml 1 M HEPES Buffer—Sigma H-0887; 2 μl neat 2-Mercaptoethanol—Sigma M-3148.

225 cm² Cell Culture Flask Tissue Culture Treated—Costar (cat no: 3001); Cedex innovatis Cell Counter; Innovatis sample cups (cat no: 600050000); Incubator set @ 37° C., 5% CO₂ humidified; Denley BR401 Refrigerated Centrifuge.

Assay Reagents:

Assay buffer: RPMI (10% FBS); RPMI—Gibco BRL (cat no: 31870-025); FBS—Sigma (cat no: F7524).

Wash buffer: 0.5% BSA/PBS; BSA—Albumin, Bovine Fraction V—Sigma A4503; Dulbecco's phosphate buffered saline (PBS) without Ca²⁺ and Mg²⁺—Gibco BRL (cat no: 14190-094).

Consumables: DMSO tissue culture grade—Sigma (cat no: D-8418); Assay tubes: Micro centrifuge tubes—Costar (cat no: 3621); FACS tubes: Falcon tubes—Becton Dickinson (cat no: 352054); Labsystems Finn pipettes Thermo Life Sciences (cat nos: 4500/090/050); 1 ml Pipette Tips—Thermo Life Sciences (cat no: 9401103); 250 μl Pipette Tips—Thermo Life Sciences (cat no: 9400263); LMS Cooled incubator; Becton Dickinson FACScalibur—Cell Quest Software; 1% formaldehyde/PBS: Formaldehyde—Sigma cat no: F1635, Dulbecco's phosphate buffered saline (PBS) without Ca²⁺ and Mg²⁺—Gibco BRL (cat no: 14190-094); IEC Micromax RF centrifuge; RANTES—R & D Systems (cat no: 278-RN-010); Mouse anti-human CCR5 monoclonal antibodies: 2D7—Pharmingen (cat no: 36461A); Isotype control antibodies: Mouse IgG2a—Pharmingen (cat no: 33031 A); Secondary PE labelled antibody: PE labelled goat anti-mouse antibody—Sigma (cat no: P9287).

Data Analysis

The Cell Quest software used for acquisition of fluorescence data was also used for data analysis. Mean fluorescence values were determined for the cell population from each assay sample. The decrease in mean fluorescence due to RANTES-mediated internalisation was measured relative to vehicle exposed 2D7 antibody control. This enabled the calculation of antagonist-dependent inhibition of RANTES mediated CCR5 internalisation, and subsequently comparison of functional occupancy by various CCR5 antagonists.

Results

The functional occupancy as measured by Compound B-dependent inhibition of RANTES-mediated CCR5 internalisation by 300.19 cells is depicted by FIG. 1. RANTES-mediated CCR5 internalisation was apparent as observed by the shift in fluorescence upon following incubation of 300.19 cells with chemokine. Inhibition of this internalisation was observed at all concentrations tested, as shown by the overlay in fluorescence for incubations where Compound B was present throughout the assay (no wash), compared to the fluorescence profile for CCR5 (as recognised by antibody 2D7) in the vehicle control. Retention of this inhibition (i.e., functional CCR5 occupancy) was observed following removal of Compound B by cell washing. Dynamic retention of inhibition was observed to varying levels at all concentrations following a 1.5 hour rolling incubation post Compound B removal.

These results highlight the dynamic retention of Compound B-dependent inhibition of RANTES-mediated uptake of CCR5 on 300.19 cells, and therefore show that functional occupancy of CCR5 by this antagonist can be retained over time. Quantification of this functional occupancy was undertaken by measuring the geometric mean fluorescence signal for each assay and calculating the percent inhibition of RANTES-mediated internalisation compared to uninhibited vehicle control. This quantification was undertaken for compounds B, C and D, to determine their functional occupancy in this assay, and demonstrate the usefulness of the assay for the identification of compounds with slow functional offset. The results of this quantification are shown in FIG. 3. The percent inhibition under each test condition are plotted on the bar chart and listed in the attached table. Where the results are average values from more than one experiment, error bars indicate the range.

As described above, compound B dissociates slowly in these experiments, whereas compound C dissociates more rapidly. In addition, it is apparent that compound C does not inhibit receptor update completely even at concentrations well in excess of its receptor IC₅₀. This is consistent with offset from CCR5 as measured by radioligand dissociation studies (see Example 3). Compound B dissociates slowly from CCR5 upon the addition of excess unlabelled compound (t_{1/2}=3.5 hours, FIG. 7). Conversely, Compound C showed relatively rapid dissociation in parallel radioligand dissociation studies (t_{1/2}=15 minutes, FIG. 8). According to the data shown in FIG. 3, compound D is a slow offset inhibitor of CCR5.

Conclusion

This FACS assay is capable of differentiating compounds based upon their dynamic occupancy of CCR5 and can be used to identify both slow and fast CCR5 offset compounds.

2. Functional Occupancy of CCR5 on Peripheral Blood Mononuclear Cells as Measured by Inhibition of MIP-1β-Mediated CCR5 Internalisation by FACS Analysis

MIP-1β mediated CCR5 internalisation (% occupancy) was assessed in CD4 T lymphocytes prepared from whole blood citrate CPT (cell preparation tubes) samples taken from healthy volunteers participating in a clinical study designed to investigate the administration of multiple oral doses of Compound A.

Compound A is a CCR5 antagonist and prevents MIP-1β binding to and subsequently internalising the CCR5 receptor. The difference in CCR5 expression on the cell surface between stabiliser treated (total CCR5) and untreated (maximum internalisation) peripheral blood mononuclear cells subjected to MIP-1β challenge will give an estimate of the proportion of free CCR5 present on the cell surface at any given plasma concentration of Compound A. This data can then be used to estimate the degree of receptor occupancy obtained at different doses of Compound A.

Methods

Single dose administrations (3 mg, 10 mg, 25 mg and 100 mg) of Compound A followed by the same doses, i.e., 3 mg, 10 mg, 25 mg and 100 mg, respectively, as multiple oral doses, b.d. for 10 days or placebo were investigated in healthy male subjects aged 18-45 years.

Subjects were allocated to one of the six cohorts (data from Cohorts 1, 4 and 6 only are illustrated here). Within each of these six cohorts, subjects were assigned to receive either Compound A at the appropriate dosing regimen or to receive placebo. The cohorts are:

Cohort 1: 100 mg b.d. Compound A or Placebo;

Cohort 4: 25 mg b.d. Compound A or Placebo;

Cohort 6: 3 mg b.d. or 10 mg b.d. Compound A or Placebo.

Prior to dosing, a blood sample was taken for tests including laboratory safety tests and genotyping evaluation. Each subject received Compound A or placebo on day 1 between 08:00 and 10:00 hours. No dose was given on day 2. A solution containing Compound A was taken by the subject while sitting or standing with water for a total volume of 250 ml. Blood samples were taken pre-dose and at intervals over the next 48 hours post-dose (day 3), as described below.

On days 3-11 subjects were dosed with Compound A or placebo between 08:00 and 10:00 hours in the morning and 20:00 and 22:00 in the evening according to the same procedures as on day 1. Each day a blood sample was taken immediately prior to the administration of the first dose of Compound A for the determination of trough plasma concentrations of Compound A.

On day 12, Compound A or placebo was dosed according to the same procedures as day 1; subjects received only one dose on day 12. Blood was taken pre-dose and at intervals over the next 72 hours post-dose.

Subjects were discharged at 24 hours post last dose but returned for a follow-up physical examination 7-10 days following the final dose.

Blood samples from subjects in Cohort 1 were taken (into 4 ml citrate CPT tubes) at the following times:

0 (Baseline), 4, 12, 24 and 48 h post 100 mg sd. Compound A dose on Day 1.

Further samples were taken on Day 12 at time 0 (Baseline) and at 24, 48, 59, 72, 97 and 120 h post 100 mg sd. Compound A dose.

For subjects in cohort 4, additional samples were collected at 8, 18, 52 and 64 h post 25 mg sd. Compound A on Day 1. On Day 12, additional samples were collected at 144, 168 and 240 h post 25 mg sd. Compound A dose.

For subjects in Cohort 6, samples were obtained at time 0 (Baseline) and at 4, 8, 12, 18, 24 and 48 h post 3 or 10 mg sd. Compound A dose on Day 1; 4 and 16 h post-dose Day 3; 2 h post-dose on Days 4 to 11 post 3 or 10 mg b.i.d. Compound A dose and then on Day 12 at time 0 (Baseline) and at 8, 12, 24, 48, 96 and 120 and 144 h post 3 or 10 mg sd. Compound A dose.

Prior to the first dose of Compound A or placebo, a 6 ml EDTA blood sample was also collected.

The residual CCR5 receptor expression on CD4 positive lymphocyte populations was determined by flow cytometric analysis of processed sodium citrate CPT anti-coagulated whole blood samples.

Materials

Sodium Citrate CPT (4 ml draw) blood tubes (Becton Dickinson. Cat no. 362760) Sample Processing tubes (12×75 mm polystyrene round bottomed) and caps (push fit) (Sarstedt Ltd, Cat nos. 55.476 and 65.809.504 respectively)

Reagents

10× concentrated PBS; 10% Paraformaldehyde; 10% Sodium Azide solution; lyophilized BSA; MIP-1β working solution (aliquots stored frozen at −70° C.; CCR5 Stabilising Solution (Contains 600 nM Compound A in PBS); Stabilising Control Solution (PBS); CCR5 and IgG Isotope Control Antibody cocktails (Store at 2-8° C.)

Reagent Preparation

1×PBS (Stable 1 month 2-8° C.)

100 mL 10× PBS

899 mL Deionized H₂O

1 mL 10% Sodium Azide solution in DI H₂O

pH 7.4

1× PBS with 1% BSA (Stable 2 weeks 2-8° C.)

1 L 1×PBS

10 g BSA lyophilized

pH 7.4

1% Paraformaldehyde (Stable 2 weeks 2-8° C.)

180 mL 1×PBS

20 mL 10% Paraformaldehyde

0.5% Paraformaldehyde (Stable 2 weeks 2-8° C.)

190 mL 1×PBS

10 mL 10% Paraformaldehyde

Preparation of CCR5 Stabilising Solution

The control stabilising solution is PBS.

1. Stock solution of 10 mM Compound A in DMSO;

2. Dilute stock solution 1:500 with 1×PBS to create a 20 μM solution (2 μl/ml PBS);

3. Dilute 20 μM solution 1:20 with 1×PBS to create a 1000 nM (1 μM) solution;

4. Dilute 100 nM solution 3:2 with 1×PBS to create a 600 nM solution.

600 nm drug solution (Stabilizing Solution) should be added 1:6 in plasma to achieve a final concentration of 100 nM. As per protocol, stabilizing solution should be added at 50 μL to 250 μL of cell enriched plasma. Control stabilising solution should be added in similar proportions to the relevant tube. The stabilising solution should be made up fresh for each cohort and vortexed prior to use to ensure that the drug is in solution.

Processing of Samples

1. Lymphocyte rich plasma was isolated by centrifugation at 1550 g for 25 mins (RT) in a swing out rotor.

2. The buffy coat layer of cells was re-suspended in the plasma by gently inverting tube several times (×5).

3. 250 μL of cell enriched plasma was pipetted into 3 separately labelled 12×75 mm polystyrene round-bottomed tubes. [Tube 1 (Control), Tube 2 (Total CCR5), and Tube 3 (CCR5 MIP1β)].

4. 50 μl of CCR5 Stabilising Solution was added to tube 2, while 50 μl of control stabilizing solution was added to tubes 1 and 3 add, before briefly vortexing on a medium setting for 2 seconds and incubating at 37° C. for 30 minutes.

5. All tubes were centrifuged at 400×g for 5 minutes in a swing out rotor to isolate cells and the supernatant then decanted.

6. To all tubes, 15 μL of MIP-1β working solution was added, then gently vortexed on a medium setting for 2 seconds to re-suspend pellet in fluid. The tubes were then incubated uncapped for 45 minutes at 37° C. in a 5% CO₂, 98% humidity atmosphere.

7. 1 ml of 0.5% paraformaldehyde in PBS was added to each tube, vortexed on a medium setting for 2 seconds and incubated for 10 minutes in the dark at room temperature.

8. 2 mL of PBS with BSA was then added to all tubes, the tubes centrifuged at 400×g for 5 minutes in a swing out rotor to pellet cells before decanting the supernatant.

9. Antibody reagents (50 μl) were added in the following manner:

To Tube 1—MsIgG R-phycoerythrin (PE) and CD4-Fluorescein (FITC) (Control) was added

To Tube 2 & 3—CCR5-PE and CD4-FITC (Total CCR5 and CCR5 MIP1β) was added.

10. All tubes were then vortexed on a medium setting for 2 seconds and incubated for 20 mins in the dark at room temperature.

11. Added 2 mL of PBS with BSA to all tubes. Centrifuged at 400×g for 5 minutes. Decanted supernatant, then added 0.5 ml of 1% Paraformaldehyde in PBS and vortexed on a medium setting for 2 seconds to re-suspend pellet.

12. Finally, tubes were capped appropriately and stored at 2-8° C. until analysis.

Results

The mean percentage occupancy of CCR5 receptors on the surface of CD4 T cells prepared from whole blood (CPT) samples and corresponding standard deviations (SD) at each nominal time point for healthy male subjects receiving a single oral dose of Compound A or placebo on day 1 followed by b.i.d. doses on days 3 to 12 are given in Table 1.

TABLE 1

| Time (h) | | | | | | | | | | |
|---|---|---|---|---|---|---|---|---|---|---|
| Post-dose | Mean | SD | Mean | SD | Mean | SD | Mean | SD | Mean | SD |
| | Cohort 1 | | Cohort 4 | | Cohort 6 | | Cohort 6 | | Cohort 1 | |
| | Day 1 | | Day 1 | | Day 1 | | Day 1 | | | |
| | (100 mg) | | (25 mg) | | (3 mg) | | (10 mg) | | (Placebo) | |
| 0 | 30.2 | 10.0 | 23.3 | 8.9 | 38.0 | 8.1 | 30.4 | 6.6 | 21.4 | 12.7 |
| 4 | 98.5 | 11.7 | 93.4 | 4.8 | 86.2 | 20.6 | 94.4 | 7.1 | 27.9 | 6.0 |
| 8 | NS | NS | 95.3 | 10.6 | 75.8 | 15.5 | 81.4 | 11.1 | 21.6 | 6.5 |
| 12 | 98.4 | 12.1 | 87.6 | 8.3 | 69.8 | 4.2 | 84.4 | 16.8 | 26.4 | 4.6 |
| 18 | NS | NS | 90.5 | 10.1 | 60.4 | 12.9 | 85.4 | 20.4 | 25.8 | 11.6 |
| 24 | 94.7 | 5.2 | 83.9 | 8.6 | 58.6 | 9.0 | 76.8 | 7.6 | 34.1 | 4.9 |
| 48 | 92.4 | 8.9 | 76.8 | 15.9 | 48.6 | 10.2 | 56.0 | 5.7 | 34.9 | 11.4 |
| 52 | NS | NS | 93.5 | 23.0 | NS | NS | NS | NS | 23.0 | 12.3 |
| 64 | NS | NS | 91.8 | 71 | NS | NS | NS | NS | 35.0 | 36 |
| | Cohort 1 | | Cohort 4 | | Cohort 6 | | Cohort 6 | | | |
| | Day 12 | | Day 12 | | Day 12 | | Day 12 | | Day 12 | |
| | (100 mg) | | (25 mg) | | (3 mg) | | (10 mg) | | (Placebo) | |
| 0 | 97.2 | 6.1 | 93.0 | 5.9 | 70.7 | 8.4 | 82.4 | 18.4 | 27.6 | 9.6 |
| 8 | NS | NS | NS | NS | 76.0 | 4.5 | 91.4 | 4.2 | 33.0 | 4.2 |
| 12 | NS | NS | NS | NS | 73.3 | 3.8 | 86.6 | 4.0 | 35.5 | 3.5 |
| 24 | 90.9 | 25.5 | 90.5 | 7.5 | 67.8 | 8.5 | 93.8 | 21.1 | 37.3 | 5.2 |
| 32 | NS | NS | NS | NS | (53.0) | — | NS | NS | NS | NS |
| 36 | NS | NS | NS | NS | (54.0) | — | NS | NS | NS | NS |
| 48 | 94.0 | 14.7 | 84.6 | 7.9 | 63.0 | 16.6 | 74.6 | 8.9 | 26.4 | 5.8 |
| 59 | 87.6 | 7.4 | NS | NS | NS | NS | NS | NS | 35.7 | 5.7 |
| 72 | 86.3 | 6.6 | 77.5 | 9.9 | (45.0) | — | NS | NS | 31.0 | 15.8 |
| 96 | 83.4 | 9.4 | 68.9 | 6.5 | 41.8 | 8.8 | 46.4 | 5.7 | 28.7 | 6.4 |
| 120 | 77.1 | 11.9 | 55.9 | 8.6 | 31.6 | 6.1 | 34.6 | 6.8 | 29.5 | 4.3 |
| 144 | NS | NS | 54.7 | 5.5 | 41.2 | 9.0 | 34.5 | 8.3 | 25.6 | 3.8 |
| 168 | NS | NS | 47.8 | 6.5 | (45.0) | — | NS | NS | 25.7 | 2.3 |

Codes:
NS:No samples taken at this point.
—:Insufficient data for calculation of SD

Conclusion

CCR5 occupancy was related to dose. Volunteers receiving 100 mg b.i.d. demonstrated CCR5 receptor occupancy in excess of 90% throughout the dosing period, while in those subjects receiving 3 mg, mean receptor saturation was <80%.

### EXAMPLE 3

### Determination of Residency Time of Compounds on CCR5

Physical occupancy of the CCR5 receptor by antagonists may be measured using radioligand dissociation studies. Radiolabelled antagonist is incubated to equilibrium with CCR5 expressed on HEK-293 cells or membrane preparations thereof. Dissociation, and hence physical occupancy or residence time, is subsequently measured by adding excess unlabelled antagonist to prevent re-association of dissociated radiolabelled antagonist. Thus, dissociation can be measured by counting retained radiolabel on whole cells or membranes over time following addition of unlabelled antagonist.

Methods

Cell culture: HEK-293 cells stably expressing CCR5 (produced by standard techniques) were grown in 225 cm² cell culture flasks to a confluency between 50-70% in culture medium (see Materials) at 37° C. for 2-3 days in the humidified 5% (v/v) CO₂ incubator. The cells were grown at low density because of their tendency to clump, with less expression of CCR5 at full confluency. Each 225 cm² flask used for the binding assays was washed once by removal of culture medium and replacement of 20 ml phosphate buffered saline (PBS) at room temperature. The supernatant was removed and the cells were dislodged by rapping the side of the culture flask in the presence of 10 ml binding buffer at room temperature (see Materials). The cells were placed into a 50 ml centrifuge tube. A further 10-20 ml binding buffer (room temperature) was added into the culture flask then transferred to the centrifuge tube to harvest residual cells. The cells were centrifuged at 350 g for 10 minutes at 20° C. The cells were resuspended in 3 ml binding buffer, counted in a modified Neubauer haemocytometer and resuspended to a density of 2×10⁶ cells/ml.

Membrane preparation: Each 225 cm² flask used for the binding assay was washed once by discarding the culture medium and replaced by 20 ml PBS (room temperature) and resuspended in 5-10 ml PBS (room temperature). The cells were transferred into a 50 ml centrifuge tube. A further 10-20 ml binding buffer (room temperature) was added into the culture flask then transferred to the centrifuge tube to harvest residual cells. The cells were centrifuged at 350 g for 10 minutes at 4° C. The cells were resuspended in 15 ml lysis buffer (see Materials) at room temperature, and homogenised with a Polytron handheld homogeniser (5-10 seconds on ice, 3-4 times on the high setting). The homogenate was transferred to Oakridge tubes and centrifuged in the Beckman ultracentrifuge (in T865 rotor) at 25000 rpm (40,000 g) for 30 minutes at 4° C. The supernatant was discarded and the pellet resuspended in a minimal volume of lysis buffer (room temperature). The protein concentration was estimated using the Bradford microassay for proteins and adjusted to 0.25 mg/ml in binding buffer. This enabled the addition of 12.5 g of membrane protein to be used in each radioligand binding assay

Compound preparation: 1-2 mg Compound A, B or C was dissolved in 100% dimethyl sulphoxide (DMSO) to a concentration of 10 mM and diluted in binding buffer to enable compound addition to the binding assays over a concentration range of 0.41 nM to 50 μM in situ. 20 μl of [³H] Compound A (62.5 μM, specific activity 16 Ci/mmol and radioactive concentration of 1 mCi/ml) was diluted in binding buffer to enable radioligand addition to the binding assay over a concentration range of 0.41-100 nM in situ. Similar dilutions were performed with [³H]-labelled Compounds B and C.

Dissociation studies: Binding buffer (25 μl) was added to wells of a 96 well assay plate, followed by 50 μl cells or cell membrane preparation. [³H] Compound A, B or C dilutions (25 μl) were added to designated wells to enable radioligand association. Non-labelled Compound (100×[³H] Compound concentration) was added designated wells (25 μl) in place of the binding buffer to determine non-specific radiolabel binding in the assays added prior to the radiolabel. The reaction mixtures were set up in duplicate to enable non-radiolabelled Compound to be added to one of the pair, to measure dissociation, and binding buffer to the other to act as an association control. This control was set up to account for dissociation occurring as a result of CCR5 or membrane/cell denaturation. The plates were incubated at room temperature for 1 hour post addition of [³H] Compound. This allowed radioligand binding to CCR5 to reach equilibrium. After this incubation, 25 μl non-labelled Compound dilutions were added to the dissociation wells. The well contents were harvested after incubations at room temperature up to 48 hours post-association.

For harvest of cells and membranes, the Unifilter plates were blocked by applying 25 ml blocking agent (room temperature) (see Materials) for 30 minutes. The Unifilter plates were washed once with 50 ml wash buffer (room temperature), the reactions were harvested by aspiration and the plate washed 3 times (1 ml/well) with wash buffer (room temperature) on the Packard Harvester. The Unifilter plate was allowed to dry overnight. Microscint 0 (50 μl—room temperature) was added to each well and a TopSeal cover placed over each plate. The plates were read (1 minute per well) on the NXT Packard TopCount Scintillation Counter.

Materials

Growth Medium: 1×500 ml Dulbecco's Modified Eagle's Medium (DMEM) with sodium pyruvate, without L-glutamine, with pyridoxine—Gibco BRL (cat no: 21969-035) containing 5 ml 200 mM L-Glutamine—Gibco BRL (cat no: 25030-024); 5 ml Penicillin/Streptomycin (100U/ml Pen/10 mg/ml Strep)—Sigma (cat no: P-7539); 50 ml foetal calf serum (FCS)—PAA Laboratories, Austria (cat no: Al 5-042); and 6.5 ml 50 mg/ml Geneticin—Gibco BRL (cat no: 10131-019)

225 cm² Cell Culture Flask Tissue Culture Treated—Costar (cat no: 3001)

Dulbecco's phosphate buffered saline (PBS) without Ca²⁺ and Mg²⁺—Gibco BRL (cat no: 14190-094).

Cell counting chamber, Improved Neubauer—Weber Scientific International (cat no: AC1000)

Binding Buffer: 50 mM HEPES in distilled water—Sigma (cat no: H-0763). 1 mM CaCl₂—Sigma (cat no: C-3881). 5 mM MgCl₂—Sigma (cat no: M-1028). 0.5% Bovine Serum Albumin (BSA)—Sigma (cat no: A-4503). Buffer adjusted to pH 7.4 (2M. HCl) and 0.2 μm filtered.

Lysis Buffer: 20 mM HEPES in distilled water—Sigma (cat no: H-0763). 1 mM CaCl₂—Sigma (cat no: C-3881). 1 tablet COMPLETE™ protease inhibitors per 50 ml lysis buffer—Boehringer Mannheim (cat no: 1697498). Buffer adjusted to pH 7.4 (2M HCl) and 0.2 μm filtered.

Wash Buffer: 10 mM HEPES in distilled water—Sigma (cat no: H-0763). 0.5M NaCl—Sigma (5M solution cat no: S-5150). 0.5% Bovine Serum Albumin (BSA)—Sigma (cat no: A-4503). Buffer adjusted to pH 7.4 (2M HCl) and 0.2 μm filtered. Stock solutions made up at 1 M (HEPES, CaCl₂ and MgCl₂) and stored at room temperature, the above buffers are prepared fresh each time. Blocking Agent: 0.3% Polyethyleneimine (PEI) in distilled water—Sigma (cat no: P-3143).

Consumables: DMSO tissue culture grade—Sigma (cat no: D-2650). Polypropylene Deep Well Blocks (1 ml/well)—Porvair (cat no: 219008). Multichannel Pipettes—Labsystems Finnpipette (cat no: 4510-000/020/030/040/050). 1 ml Pipette Tips—Sigma (cat no: P7174). 250 μl Pipette Tips—Sigma (cat no: P7049). Reagent Reservoirs for multichannel pipettes—Costar (cat no: 4870). Packard filtrate Universal Harvester (96 well head). Packard UniFilter GF/B 96 well filter plates with bottom seal—Packard (cat no: 6005177). Microscint 0—Packard (cat no: 6013611). TopSeal-A microplate press-on adhesive sealing film—Packard (cat no: 6005185). NXT Packard TopCount Scintillation Counter.

Results

As shown in FIG. 6, 50% of Compound A was still bound after 9 hours of incubation with excess unlabelled Compound A. After 3 hours, 50% of Compound B was still bound (FIG. 7), whereas 50% of Compound C was still bound after 15 minutes (FIG. 8).

### EXAMPLE 5

### Antiviral Activity

Anti-HIV activity in peripheral blood lymphocytes (PBLs) for compounds was tested by infecting these cells with HIV (isolate Ba-L) and measuring compound-dependent reduction in supernatant reverse transcriptase (RT) activity following incubation for 5 days.

Methods

The HIV-1 Ba-L and IIIB viruses used in the antiviral assays were obtained from the AIDS Reagent Project, NIBSC, Potters Bar, Herts, UK (repository references ARP118 and ARP101). The virus was stored in 1 ml vials at −80° C. For expansion of HIV-Ba-L virus stock (1 ml) was removed from −80° C. storage, and rapidly thawed in a 37° C. incubator for 10 minutes. Virus stock (0.5 ml) was placed in a labelled well of a 24 well plate with 1 ml of fresh PBLs (pre-stimulated 3 days with phytohaemagglutinin (PHA) at a final concentration of 1.5 μg/ml) at a cell density of 1.0×10⁷/ml in complete RPMI 1640 growth medium (supplemented with 10% FCS, 2 mM L-glutamine, and 1U/ml penicillin, 0.1 mg/ml streptomycin). The 24-well plate was incubated at 37° C. in a humidified 5%(v/v) CO₂ incubator for 1 hour to allow infection. After 1 hour, the cells and supernatant from this plate were centrifuged at 225 g for 10 minutes at 25° C., and the supernatant discarded. The cells were suspended in 10 ml of RPMI 1640 growth medium at room temperature and transferred to a 25 cm² tissue culture flask. IL-2 was added to a final concentration of 10 ng/ml. The infected PBL culture was incubated in an upright position at 37° C. in humidified 5%(v/v) CO₂ incubator for 3 days. At day 3, 5 mls of fresh RPMI 1640 growth medium were added together with IL-2 at a final concentration of 10 ng/ml.

After 7 days the infected cells were transferred to an 80 cm² tissue culture flask. A further 7 ml of IL-2-stimulated PBLs (1.0×10⁷ cells/ml) in RPMI 1640 growth medium was added, and incubated in a humidified 5%(v/v) CO₂ incubator until day 10. Fresh RPMI 1640 growth medium (25 ml), containing 10 ng/ml IL-2, was added at day 10 followed by gentle mixing. The culture was separated into two top 80 cm² tissue culture flasks, and incubated at 37° C. in a humidified 5%(v/v) CO₂ incubator until day 15.

Viral expansion was measured by reverse transcriptase (RT assay), and counts of ≧4000 cpm per 20 μl supernatant from the RT assay were deemed as active. Cells were expanded further by dividing the infected culture in to fresh 80 cm² tissue culture flasks, followed by the addition of 13 ml of fresh IL-2-stimulated PBLs (1.0×10⁷ cells/ml)/ml. The flasks were incubated at 37° C. in a humidified atmosphere of 5% CO₂ until day 18. At day 18, 20 ml of RPMI 1640 growth medium (containing IL-2 at long/ml) was added. The cultures were incubated at 37° C. in a humidified atmosphere of 5% CO₂ until day 23. Virus samples were deemed active if RT counts in the supernatant (20 μl) were >5000 cpm at day 23. These supernatants were clarified by centrifugation at 850 g for 10 minutes at 250° C., and the resultant supernatant mixed, and aliquoted into 4 ml and 1 ml cryotubes for storage at −80° C.

Virus Titration

The tissue culture infectious dose 50 (TCID₅₀—assumed number of viral particles required to infect 50% of cells) was calculated for the HIV Ba-L stock (harvested Dec. 17, 1999), by the Reed and Muench equation ((1938) Am. J. Hyg. 27, 493-497), (using RT as a positive marker for HIV infection). Titration of virus stock HIV-1 Ba-L (harvest Dec. 17, 1999) produced a TCID₅₀5.76×10⁴/ml using this method. All titrations were performed using freeze-thawed virus stocks, in fresh-pooled PHA-stimulated PBLs. To prepare PBLs, cells were pelleted by centrifugation for 10 minutes at 500 g and 25° C., checked for viability by removing 100 μl of cells, followed by the addition of 100 μl of cell dissociation solution (see Materials) and 100 μl of 0.4%(v/v) Trypan Blue. Viable (uncoloured) cells were counted in a Kova counting chamber (see Materials). Only cell suspensions showing >95% viability progressed in assays. Viable cells were resuspended to a density of 2.0×10⁶ cells/ml in RPMI growth medium (10 ng/ml IL-2 added for PBL cultures). Expanded frozen HIV Ba-L stock (2×1 ml) were rapidly thawed in a 37° C. incubator for 10 minutes. One sample was progressed for titration assessment and the other progressed for antiviral testing in parallel.

For titration, 25 μl of thawed expanded virus stock, was progressed through 8 x 0.5 log serial dilutions in RPMI growth medium. Each viral dilution (20 μl) was transferred to a 96-well assay plate together with 180 μl of PBLs. The assay plate was incubated at 37° C. in humidified atmosphere of 5% (v/v) CO₂ for 5 days, after which 100 μl of supernatant was harvested and tested for RT activity, to enable TCID₅₀ determination. The titred virus was deemed to be positive with respect to virus infectivity when RT counts were greater (>2× standard deviations) than the uninfected cell counts. Viral titre was calculated using the method reported by Reed and Muench ((1938) Am. J. Hyg. 27, 493497). This enables the standardisation of viral input for the antiviral assays and subsequent antiviral potency testing of the compounds. The TCID₅₀ for HIV-Ba-L in the antiviral assays in this study was 5.76×10⁴/ml.

Preparation of PBL Cultures with Monocytes

The North London Blood Transfusion Centre supplied single donor buffy coats containing PBLs, from HIV and HBV sera-negative donors. Serological status was determined by North London Blood Transfusion services. PBLs were prepared from 4 buffy coat samples that were individually separated and expanded in culture. Each of the 4 buffy coats (50 ml) were transferred into an 80 cm² tissue culture flask with an equal volume of sterile phosphate-buffered saline (PBS). Aliquots (25 ml) of cell suspension were gently layered onto 25 ml Ficoll-Paque in separate 50 ml centrifuge tubes. Following centrifugation for 30 min at 1000 g at 25° C., the PBL layer was removed by pipetting from between the erythrocyte and plasma layers. The separated PBLs were transferred to fresh centrifuge tubes and washed twice with PBS (4° C.) and centrifugation for 10 minutes at 850 g at 4° C. Contaminating erythrocytes were removed by adding 9 ml sterile water to the PBL pellet together with ×10 Hanks Balanced Salt Solution (see Materials). PBS (4° C.) was added to give a final volume of 45 ml. The PBLs were centrifuged for 10 min at 500 g at 4° C. The pellet was suspended in 30 ml RPMI 1640 growth medium (room temperature).

Cell viability was checked as described above, and only cell suspensions showing >95% viability were processed for antiviral assay. The cell suspension was adjusted to a density of 1×10⁶ cells per ml by the addition of RPMI 1640 growth medium supplemented 1.5 μg/ml PHA. The cells (50 ml) were transferred to 80 cm² tissue culture and incubated for 3 days at 37° C. in a humidified 5% CO₂ (v/v) incubator, in preparation for antiviral assay.

Separation and Growth of PBLs without Monocytes

The PBLs were prepared and tested for viability by Trypan Blue staining as described above. PBL preparations with >95% viability were transferred to 80 cm² culture flasks in a total volume of 50 ml and cell density of 1×10⁶/ml. The flasks were incubated for 1 hour at 37° C. in a humidified 5% CO₂ (v/v) incubator to facilitate monocyte adhesion to the flask surface. The non-adherent PBLs were carefully decanted into 50 ml centrifuge tubes and pelleted by centrifugation at 500 g for 10 min at 25° C. The PBL pellet was adjusted to a density of 1×10⁶ cells per ml by the addition of fresh RPMI 1640 growth medium containing 1.5 μg/ml PHA. The cells (50 ml) were transferred to 80 cm² tissue culture and incubated for 3 days at 37° C. in a humidified 5% CO₂ (v/v) incubator, in preparation for antiviral assay.

Antiviral Assay in PBLs (with or without Monocytes)

The PHA-stimulated cell cultures were dispersed by gentle shaking and transferred to a sterile 50 ml centrifuge tube. The cells were pelleted by centrifugation for 10 minutes at 500 g at 25° C. and resuspended in 50 ml RPMI growth medium. Cell viability was checked as before. Only cell suspensions showing >95% viability were processed for antiviral assay. For cell infection, expanded Ba-L stock of known titre was added to the PBL cell pellet, at a ratio of 250 μl stock per 1.0×10⁶ cells, TCID₅₀5.76×10⁴/ml followed by the addition of 125 μl RPMI growth medium (containing 10 ng/ml IL-2) per 1.0×10⁶ cells. The cells were then incubated for 1 hat 37° C. in a humidified 5%(v/v) CO₂ incubator, followed by centrifugation at 500 g for 10 minutes at 25° C. The cell pellet was resuspended in RPMI growth medium (containing 10 ng/ml IL-2) at a density of 2.0×10⁵/ml. 1.8 ml aliquots of infected cell suspension were transferred to a 24-well assay plate, containing 200 μl of compound. The final concentration range of compound in the assay plate was 0-100 nM (0.1% DMSO). The assay plate was placed in a humidified 5% CO₂ (v/v) incubator at 37° C. for 5 days.

Non-infected PBL controls were processed in parallel, together with a positive control containing the anti-HIV inhibitor RANTES tested over a 0-100 nM and 0-33 nM concentration range. Following the 5 day incubation, 200 μl of supernatant from each well was transferred to a 96 well plate, for quantification of virus yield by reverse transcriptase activity, and subsequent determination of the compounds' antiviral potency.

Reverse Transcriptase Assay

Quantification of viral yield by direct measurement RT activity in HIV infected cell cultures was performed using a scintillation proximity assay kit from Amersham (see Materials). RT activity was assayed by monitoring [³H] TTP incorporation into biotin-labelled DNA primer linked to the surface of the SPA beads. Reagent volumes for RT assay depended on the number of assays to be run. To enable 100 assays, 1 ml of stock [³H] TTP (3700 KBq) was diluted in 2 ml assay kit buffer, and transferred to an 80 cm² culture flask together with 5 ml sterile water and 1 ml SPA bead from assay kit. This solution was mixed thoroughly and 80 μl aliquots were added to each well in the RT reaction plate, followed by 20 μl of the HIV-infected PBL supernatants. The reaction plates were sealed and placed in a 37° C. incubator for 1 hour.

To stop the RT reaction, 100 μl of assay kit stop buffer (2% (w/v) SDS, pH 8) was added. The plates were centrifuged at 250 g for 10 minutes at 4° C. Scintillation was measured for each well over a 60 second period using a 1450 Microbeta liquid scintillation counter, following a 20 minute incubation period in situ to enable bead settlement. Background counts for uninfected control samples were subtracted from infected sample counts.

A standard curve for RT activity was generated by running purified recombinant RT samples in parallel to the HIV-infected cell samples, to ensure RT levels from the supernatants were in the linear range for the assay. For the RT standard curve, 20 μl of stock recombinant reverse transcriptase (10U/μl—see Materials) was defrosted and diluted with 9980 μl complete RPMI 1640 medium 4° C. The enzyme was stored at −20° C. until used to produce serial dilutions in complete RPMI 1640 medium at 4° C. (20, 10, 5, 2.5, 1.25, 0.62, 0.31 and 0.15 mU/μl) for a standard curve. Each enzyme dilution (20 μl) was sampled in duplicate in the RT assay.

Cytotoxicity Assay

Potential non-specific cytotoxicity of compounds in PBLs was investigated as a possible cause for apparent antiviral activity. The assay was performed in 96-well format, using a calorimetric cell proliferation kit from Promega (see Materials). In this assay, the tetrazolium compound 3-(4,5-dimethylthiazol-2-yl)-5-(3-carboxymethoxyphenyl)-2-(4-sulfophenyl)-2H-tetrazolium (MTS) in combination with the electron-coupling reagent phenazine methosulphate (PMS), is reduced by dehydrogenase activity in viable cells to generate formazan. The absorbance of the formazan at 492 nm is directly proportional to the density of living cells in the culture. This assay was performed on uninfected PBLs separately cultured in parallel to those used in the antiviral assays. The PBLs were prepared for the cytotoxicity assay as described for the antiviral assays, prior to HIV infection. The PBLs were suspended in fresh culture medium to a final density of 2×10⁵ cells/ml. The cell suspensions (180 μl) were transferred to a 96-well plate, in addition to 20 μl of compound to yield a final concentration range of 0-10 μM in situ (0.1%(v/v DMSO). The assay plates were incubated at 37° C. in a humidified 5% CO₂ (v/v) incubator for 5 days. Following this incubation 40 μl of MTS/PMS mixture from the cytotoxicity assay kit was added to each well, and incubated in the dark for 3 hours at 37° C. humidified 5% (v/v) CO₂ incubator. The plates were then shaken by hand, and absorbance at 492 nm measured spectrophotometrically to assess the viability of the cells and non-specific cytotoxicity of the compounds.

Data Analysis

Antiviral activity was determined by plotting a graph of percent reduction in RT activity or p24 levels (relative to no compound control), against compound concentration (log scale). The IC₅₀ and IC₉₀ values were determined using the fit curve option of Microsoft Excel for duplicate datapoints at each concentration. The IC₅₀ and IC₉₀ values for independently prepared concentration ranges tested of compound were determined and the geometric mean calculated using standard software packages. These experiments were independently repeated with different preparations of PBL cells, and the overall geometric mean IC₅₀ and IC₉₀ values, together with the 95% confidence intervals were calculated. The same analysis was performed for the antiviral standard RANTES. Individual IC₅₀ and IC₉₀ values which fell outside the concentration range of compound, or RANTES were not included in further calculation for geometric mean IC₅₀ or IC₉₀.

Materials

HIV-1 strain Ba-L: a cell free supernatant was obtained from AIDS Reagent Project (NIBSC, Potters Bar, Herts, UK.).

RANTES: Obtained from R & D systems (cat no. 278-RN-010 or 278-RN-050)

Cell dissociation solution: (Sigma 1×conc. C5789 lot 59H0890)

Cell culture medium: RPMI medium was obtained from Sigma (cat no: R0883), L-glutamine from Life technologies (cat no: 25030-024), FCS from Sigma for PBL cells (cat no: F2524 batch 77H3399), Penicillin & streptomycin from Life Technologies (cat no 15140-123).

Additives for PBL Culture medium: PHA suspended in sterile water to 1 mg/ml, and used at 1.5 μg/ml final concentration, Murex-Abbott Laboratories (cat no: HA16). IL-2, human recombinant dissolved in 4 mM HCl plus 0.1% (v/v) FCS to 2 μg/ml (approx. 6000 U/ml) and used at 10 ng/ml final concentration.—R&D Systems (cat no: 202-IL-050)

PBL isolation reagents: Buffy coats supplied by North London Blood Transfusion Centre (Colindale Centre, Ficoll-Paque solution from Pharmacia Biotech (Cat no: 17-0840-02), PBS from Sigma (Cat no: D8537), Sterile distilled water from Sigma (Cat no: W-3500)

Reverse transcriptase assay kit: Quan-T-RT assay system from Amersham Life Science (cat no: TRK 1022). Recombinant HIV reverse transcriptase, supplied as 200U (10U/μl) from Amersham, (cat no: T3610Y).

Cytotoxicity assay kit: CellTiter 96® AQᵤₑₒᵤₛ Non-Radioactive Assay—Promega (cat no: G5430)

Consumables: 24 well sterile flat bottom Falcon plates—Becton Dickinson (cat no: 3047). 96 well sterile flat bottom Falcon plates—Becton Dickinson (cat no: 3072). 96 square well plate polypropylene 2 ml capacity per well sterile (Beckman Coulter cat no 609681). Cell counting chamber-Kova Biostat Diagnostics (cat no: 887144).

Results

In the antiviral assay, Compound D has an IC₉₀ of 1 nM, Compound A and Compound B have an IC₉₀ in the antiviral assay of around 2 nM, whereas Compound C has a potency in the antiviral assay of >100 nM.

Conclusion

Although Compounds A to D have very similar binding affinity to CCR5, the antiviral effect is markedly improved for ligands that have a slow dissociation rate, or a long residence time on CCR5. Therefore, the residence time of a ligand on CCR5 may be used as a predictor for the potency of the ligands for functional activity, in this case antiviral activity, and predicts clinical efficacy for the ligand as an antiviral agent.

### EXAMPLE 6

### Effect of Short-Term Monotherapy with Compound A on Viral Load in HIV-Infected Patients

For standard antiretroviral agents that target the virus directly, there is a direct relationship between antiviral effects (as measured by a decrease in plasma viral load) and plasma drug concentrations. Likely clinically efficacious dose ranges can therefore be selected based on in vitro antiviral data i.e., IC₅₀ and IC₉₀. However, an argument can be made that for this approach, saturation of the CCR5 receptor could be a better predictor of efficacy and may not be directly related to plasma drug concentrations.

Data from healthy volunteer studies demonstrated that even at a dose of 25 mg where the drug concentrations rapidly fell to well below the antiviral IC₉₀, essentially complete saturation of the receptor was achieved for >24 hours post-dose. The rationale for this study was therefore, inter alia, to determine whether saturation of the CCR5 receptor correlates with antiviral effects in vivo.

Methods

Twenty-four asymptomatic HIV seropositive male subjects with CD4 T cell count >250 cells/mm³ and plasma viral load >5000 copies/ml received Compound A 25 mg o.d., 100 mg b.i.d. or placebo for 10 days. Main exclusion criteria were liver enzyme abnormalities, CD4 T cell count <250 cells/mm³, viral load <5000 copies/ml and the presence of CXCR4 or dual tropic HIV.

Receptor Saturation

Receptor saturation was evaluated using the CCR5/MIP-1β, internalisation assay described above at the following time points:

Day 1: 0 (pre-morning dose) and 4 hours post dose

Day 5: pre-morning dose

Day 10: pre-dose and 12 hours post-dose

Days 11, 12, 13, 15, 19 and 40

Viral Load

Viral load was determined using an RT-PCR (Roche Amplicor v1.5) assay with a lower limit of detection of 400 copies/ml as standard. For samples with a reading of <400 copies/ml, the ultrasensitive method with a lower limit of detection of 50 copies/ml was used. Samples were taken at the following timepoints:

Day 1: 0 (pre-morning dose) and 4, 12 and 18 hours post-dose

Day 2-10: 0 (pre-morning dose)

Days 11, 12, 13, 15, 19, 22, 25 and 40

Results

Mean CCR5 receptor saturation in patients receiving 100 mg b.i.d. was in excess of 90% throughout the dosing period, but in subjects receiving 25 mg od mean receptor saturation fell to <80% by day 10 pre-dose (steady state). FIG. 9 demonstrates receptor saturation over time (in hours post-first dose) per dose group.

Seven of eight patients receiving 100 mg b.i.d. of Compound A showed a good viral load response with a viral load decline of >1 log ₁₀. The mean drop in viral load from baseline to day 11 in this group was 1.200 log ₁₀. One subject in the 100 mg b.i.d. dose group, in whom co-existence of viruses using CCR5 and/or CXCR4 as their entry coreceptor was demonstrated on day 1, had no response to Compound A. If this individual is excluded from the analysis the drop in viral load in the 100 mg group is 1.419 log ₁₀. In the 25 mg o.d. dose group a mean viral load reduction of 0.4251 log ₁₀ was demonstrated (Table 2). Following termination of dosing (day 11 onwards) viral load returned to baseline over time and by day 40 most patients have returned to baseline.

TABLE 2
Mean viral load change from baseline by dose group.

| | | Log₁₀ viral load change from baseline* |
|---|---|---|
| Dose | N | to day 11 |
| Placebo | 8 | −0.019 |
| 25 mg od | 8 | −0.425 |
| 100 mg b.i.d. | 8 | −1.200 |

*Baseline is calculated as the mean of the 3 pre-dose viral load measurements

Conclusions

Mean CCR5 receptor saturation in patients receiving 100 mg b.i.d. was in excess of 90% throughout the dosing period, but in subjects receiving 25 mg o.d., mean receptor saturation fell to <80% by day 10 pre-dose. Subjects receiving 100 mg b.i.d. had a mean decrease in viral load of 1.200 log ₁₀ from baseline to day 11, and there was evidence of a response in subjects receiving 25 mg o.d.

Clinical data shows that Compound A exhibited potent antiviral effects when given as short-term monotherapy and thus supports the in vitro data and its prediction of clinical efficacy.

From the examples given above, it can be seen that CCR5 residence, or occupancy, time in vitro may be correlated with receptor occupancy in vivo. More importantly, ligands identified by in vitro receptor occupancy show significant decreases in viral load both in vitro and in vivo. Thus, measurement of occupancy of a ligand on its receptor in vitro may be used to identify a ligands predicted to be efficacious in vivo.

While this theory has been tested and proved in an HIV scenario, it will be appreciated that the prediction of the clinical efficacy of putative ligands from in vitro receptor occupancy may be extended to other CCR5-mediated medical indications, including respiratory disorders, including adult respiratory distress syndrome (ARDS), bronchitis, chronic bronchitis, chronic obstructive pulmonary disease, cystic fibrosis, asthma, emphysema, rhinitis and chronic sinusitis.

Other conditions for which a correlation with CCR5 or CCR5 chemokines has been established include inflammatory bowel disease, including Crohn's disease and ulcerative colitis, multiple sclerosis, rheumatoid arthritis, graft rejection, in particular but not limited to solid organ transplants, such as heart, lung, liver, kidney and pancreas transplants (e.g. kidney and lung allografts), endometriosis, type I diabetes, renal diseases, such as glomerular disease, fibrosis, such as liver, pulmonary and renal fibosis, chronic pancreatitis, inflammatory lung conditions, encephalitis, such as HIV encephalitis, chronic heart failure, psoriasis, stroke, obesity, CNS diseases, such as AIDS related dementias and Alzheimer's Disease, anaemia, atherosclerotic plaque, atopic dermatitis, chronic pancreatitis, cancer, such as non-Hodgkin's lymphoma, Kaposi's sarcoma, melanoma and breast cancer, and pain, such as nociceptive pain and neuropathic pain (e.g. peripheral neuropathic pain).

Infectious diseases where modulation of the CCR5 receptor is implicated include acute and chronic hepatitis B Virus (HBV) and HCV infection, bubonic, septicemic, and pneumonic plague, pox virus infection, such as smallpox, toxoplasmosis infection, mycobacterium infection, trypanosomal infection such as Chagas' Disease, pneumonia, and cytosporidiosis.

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

1. A method for identifying a ligand in the treatment of a disease that responds to modulation of a receptor comprising measuring said ligand residency time on said receptor in vitro and selecting said ligand on the basis of a desired residency time on said receptor.

2. A method according to claim 1 wherein said residency time is at least 1 hour.

3. A method according to claim 1 wherein said residency time is at least 3 hours.

4. A method according to claim 1 wherein said residency time is at least 6 hours.

5. A method according to claim 1 wherein said residency time is at least 9 hours.

6. A method according to claim wherein said receptor is CCR5.

7. A method according to claim 1 wherein said ligand is a CCR5 antagonist.

8. A method according to claim 1 wherein said disease is infection by a virus.

9. A method according to claim 1 wherein said disease is HIV.

10. A method comprising measuring the receptor residence time of each of a plurality of ligands for said receptor and selecting at least one of said ligands whose residence time exceeds that of at least one other ligand.

11. A method according to claim 10 wherein said residence time is at least 1 hour.

12. A method according to claim 10 wherein said residence time is at least 3 hours.

13. A method according to claim 10 wherein said residence time is at least 6 hours.

14. A method according to claim 10 wherein said residence time is at least 9 hours.

15. A method according to claim 10 wherein said receptor is CCR5.

16. A method according to claim 10 wherein said ligand is a CCR5 antagonist.

17. A method comprising contacting a plurality of ligands for a given receptor with said receptor, measuring the receptor binding affinity and receptor residence time of each ligand, assigning to each ligand a rank value which is the product of its measure binding affinity and its receptor residence time, and selecting one or more ligands having a rank value greater than a chosen cut-off rank value.

18. A method according to claim 17 wherein said residence time is at least 1 hour.

19. A method according to claim 17 wherein said residence time is at least 3 hours.

20. A method according to claim 17 wherein said residence time is at least 6 hours.

21. A method according to claim 17 wherein said residence time is at least 9 hours.

22. A method according to claim 17 wherein said receptor is CCR5.

23. A method according to claim 17 wherein said ligand is a CCR5 antagonist.

24. A method for identifying ligands with high potency and clinical efficacy for a disease that responds to modulation of a receptor's natural function which comprises measuring the residence time of said ligands on said receptor and selecting said ligands on the basis of the desired residence time.

25. A method according to claim 24 wherein said residence time is at least 1 hour.

26. A method according to claim 24 wherein said residence time is at least 3 hours.

27. A method according to claim 24 wherein said residence time is at least 6 hours.

28. A method according to claim 24 wherein said residence time is at least 9 hours.

29. A method according to claim 24 wherein said receptor is CCR5.

30. A method according to claim 24 wherein said ligand is a CCR5 antagonist.

31. A method according to claim 24 wherein said disease is infection by a virus.

32. A method according to claim 24 wherein said disease is HIV.
